(19) United States
(12) Patent Application Publication
(10) Pub. No.: US 2011/0243863 A1
(43) Pub. Date: Oct. 6, 2011

(54) Cosmetic And/Or Pharmaceutical Formulations

(76) Inventors: Rolf Kawa, Monheim (DE); Bettina Jackwerth, Langenfeld (DE)

(21) Appl. No.: 13/124,957

(22) PCT Filed: Oct. 12, 2009
(86) PCT No.: PCT/EP2009/007430

§ 371 Date: Apr. 19, 2011

(30) Foreign Application Priority Data
Oct. 21, 2008 (DE) 10 2008 052 520.0

(51) Int. Cl. A61K 8/37; C07C 53/126; A61Q 15/00; A61Q 17/04; A61Q 19/00

(52) U.S. Cl. 424/59; 554/1; 424/65; 514/552

(57) ABSTRACT

The invention relates to cosmetic and/or pharmaceutical formulations, comprising n-octyl-n-octanoate.

The present invention relates to cosmetic and/or pharmaceutical preparations, comprising 0.1 to 80% by weight of n-octyl n-octanoate.

### PRIOR ART

In the field of cosmetic emulsions for skincare and hair care, a large number of requirements is demanded by the consumer: apart from the cleaning and care effects, which determine the intended use, value is placed on such differing parameters as highest possible dermatological compatibility, good refatting properties, elegant appearance, optimum sensory impression and storage stability. Preparations which are used for the cleaning and care of human skin and hair generally comprise, besides a series of surface-active substances, in particular oil bodies and water. The oil bodies/emollients used are, for example, hydrocarbons, ester oils, and vegetable and animal oils/fats/waxes. In order to meet the high requirements of the market with regard to sensory properties and optimal dermatological compatibility, novel oil bodies and emulsifier mixtures are being continuously developed and tested. The use of ester oils in cosmetics has been known for a long time. On account of their importance, novel processes for their preparation are also being continuously developed. It was an object of the present invention to provide novel ester oils, which are preferably liquid at 20° C., for cosmetic and/or pharmaceutical applications which have an improved profile as regards the sensory properties (lightness, “non-greasy skin feel”, softness, spreadability, absorption, distributability, oiliness) and can be incorporated into a large number of cosmetic and/or pharmaceutical formulations. Here, the hydrolysis stability of the esters and also the formulatability of the esters at low pH were also of interest. Furthermore it should also be possible to incorporate the esters both into W/O and also into O/W formulations. Furthermore, the esters should be compatible in particular with crystalline UV filters, pigments, antiperspirant salts and silicones. Furthermore, the esters should be oxidation-stable. Furthermore, the so-called “non-transfer” property is of interest especially for decorative cosmetic preparations (for example make-up). Additionally, the compatibility of the esters with preparations which comprise washing-active substances (such as e.g. shower baths, shampoos, hair conditioners) is of interest. It was also of particular interest to provide substances which can completely or partially replace silicone oil in cosmetic and/or pharmaceutical preparations. The silicone oils used in the prior art are disadvantageous inter alia on account of their bioaccumulation. It was also of particular interest to provide substances which have a low irritation potential (inter alia skin and eyes). WO 2006/097235 describes esters of 2-propyl-heptanol with linear or branched carboxylic acids. The object of the present invention was to provide improved esters compared with the prior art. It has been found that the esters of the present invention achieve this object.

### DESCRIPTION OF THE INVENTION

The cosmetic and/or pharmaceutical preparations according to the invention are light and stable cosmetic and/or pharmaceutical preparations. One subject matter of the invention relates to cosmetic and/or pharmaceutical preparations, comprising n-octyl n-octanoate. A preferred subject matter of the invention relates to cosmetic and/or pharmaceutical preparations, comprising 0.1 to 80% by weight of n-octyl n-octanoate.

n-Octyl n-octanoate

n-Octyl n-octanoate is the ester of n-octanol with n-octanoic acid and conforms to the following formula:

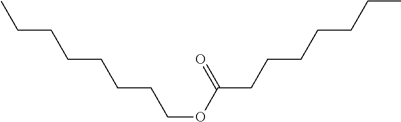

It has been found that this ester has good cosmetic properties and is particularly suitable as oil body in cosmetic and/or pharmaceutical preparations. n-Octyl n-octanoate, i.e. the ester of n-octanol and n-octanoic acid, is known as substance (CAS No. 2306-88-9), e.g. as pheromone in the animal kingdom. The cosmetic use of this substance is not described in the prior art. n-Octyl n-octanoate can be prepared by methods known to the person skilled in the art, such as e.g. esterification from alcohol and acid or transesterification from alcohol and methyl ester.

The cosmetic and/or pharmaceutical preparations according to the invention are light and stable cosmetic and/or pharmaceutical preparations, this being the case particularly when they also comprise antiperspirant/deodorant active ingredients. One subject matter of the invention therefore relates to cosmetic and/or pharmaceutical preparations, comprising n-octyl n-octanoate and at least one antiperspirant/deodorant active ingredient. One subject matter of the invention therefore relates to cosmetic and/or pharmaceutical preparations, comprising 0.1 to 80% by weight of n-octyl n-octanoate and at least one antiperspirant/deodorant active ingredient.

According to the invention, suitable antiperspirant/deodorant active ingredients are all active ingredients which counteract, conceal or eliminate body odors. Body odors are formed as a result of the action of skin bacteria on apocrine perspiration, with the formation of unpleasant smelling degradation products. Suitable antiperspirant/deodorant active ingredients are in particular compounds selected from the group consisting of antiperspirants, esterase inhibitors, bactericidic or bacteriostatic active ingredients and/or perspiration-absorbing substances.

Antiperspirants

Antiperspirants are salts of aluminum, zirconium or zinc. Such suitable antihydrotic active ingredients are, for example, aluminum chloride, aluminum chlorohydrate, aluminum dichlorohydrate, aluminum sesquichlorohydrate and complex compounds thereof e.g. with 1,2-propylene glycol. Aluminum hydroxy-allantoinate, aluminum chloride tartrate, aluminum zirconium trichlorohydrate, aluminum zirconium tetrachlorohydrate, aluminum zirconium pentachloro-hydrate and complex compounds thereof e.g. with amino acids such as glycine. Preference is given to using aluminum chlorohydrate, aluminum zirconium tetrachloro-hydrate, aluminum zirconium pentachlorohydrate and complex compounds thereof.

The preparations according to the invention can comprise the antiperspirants in amounts of from 1 to 50, preferably 5 to 30 and in particular 8 to 25% by weight—based on the total weight of the cosmetic and/or pharmaceutical preparation.

Esterase Inhibitors

In the presence of perspiration in the axillary area, extracellular enzymes—esterases, preferably proteases and/or lipases—are formed by bacteria; these cleave esters present in the perspiration and thereby release odorous substances. Suitable esterase inhibitors are preferably trialkyl citrate such as trimethyl citrate, tripropyl citrate, triisopropyl citrate, tributyl citrate and in particular triethyl citrate (Hydagen® CAT, Cognis GmbH, Dusseldorf/FRG). The substances inhibit the enzyme activity and thereby reduce the formation of odor. Further substances which are suitable as esterase inhibitors are sterol sulfates or phosphates, such as, for example, lanosterol, cholesterol, campesterol, stigmasterol and sitosterol sulfate and phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

The preparations according to the invention can comprise the esterase inhibitors in amounts of from 0.01 to 20, preferably 0.1 to 10 and in particular 0.3 to 5% by weight—based on the total weight of the cosmetic and/or pharmaceutical preparation.

Bactericidic and Bacteriostatic Active Ingredients

Typical examples of suitable bactericidic and bacteriostatic active ingredients are in particular chitosan and phenoxyethanol. 5-Chloro-2-(2,4-dichloro-phenoxy)phenol has also proven particularly effective; this is sold under the name Irgasan® by Ciba-Geigy, Basel/CH. Suitable germicidal agents are in principle all substances that are effective against Gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxy-diphenyl ether (triclosan), 4-chloro-3,5-dimethyl-phenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorhexidine, 3,4,4′-trichlorocarbanilide (TTC), antibacterial fragrances, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), N-alkylamides of salicylic acid, such as, for example, N-n-octylsalicylamide or N-n-decylsalicylamide.

The preparations according to the invention can comprise the bactericidic or bacteriostatic active ingredients in amounts of from 0.01 to 5 and preferably 0.1 to 2% by weight—based on the total weight of the cosmetic and/or pharmaceutical preparation.

Perspiration-Absorbing Substances

Suitable perspiration-absorbing substances are modified starch, such as, for example, Dry Flo Plus (National Starch), silicates, talc and other substances of similar modification which appear to be suitable for the absorption of perspiration. The preparations according to the invention can comprise the perspiration-absorbing substances in amounts of from 0.1 to 30, preferably 1 to 20 and in particular 2 to 8% by weight—based on the total weight of the cosmetic and/or pharmaceutical preparation.

The cosmetic and/or pharmaceutical preparations according to the invention are light and stable cosmetic and/or pharmaceutical preparations, this being the case particularly when they also comprise at least one UV photoprotective filter. One subject matter of the invention therefore relates to cosmetic and/or pharmaceutical preparations, comprising n-octyl n-octanoate and at least one UV photoprotective filter. One subject matter of the invention preferably relates to cosmetic and/or pharmaceutical preparations comprising 0.1 to 80% by weight of n-octyl n-octanoate and at least one UV photoprotective filter.

According to the invention, suitable UV photoprotective filters are organic substances (photoprotective filters) that are crystalline or liquid at room temperature and which are able to absorb ultraviolet rays and release the absorbed energy again in the form of longer-wave radiation, e.g. heat. UV filters may be oil-soluble or water-soluble. Typical oil-soluble UV-B filters and broadband UV A/B filters to be mentioned are, for example:
3-benzylidenecamphor or 3-benzylidenenorcamphor (Mexoryl SDS 20) and derivatives thereof, e.g. 3-(4-methylbenzylidene)camphor as described in EP 0693471 B1
3-(4′-trimethylammonium)benzylidenebornan-2-one methyl sulfate (Mexoryl SO)
3,3′-(1,4-phenylenedimethine)bis(7,7-dimethyl-2-oxobicyclo[2.2.1]heptane-1-methanesulfonic acid) and salts (Mexoryl SX)
3-(4′-sulfo)benzylidenebornan-2-one and salts (Mexoryl SL)
polymer of N-{(2 and 4)-[2-oxoborn-3-ylidene)-methyl}benzyl]acrylamide (Mexoryl SW)
2-(2H-benzotriazol-2-yl)-4-methyl-6-(2-methyl-3-(1,3,3,3-tetramethyl-1-(trimethylsilyloxy)-disiloxanyl)propyl)phenol (Mexoryl XL)
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);
esters of salicylic acid, preferably 2-ethylhexyl salicylate, 4-isopropylbenzyl salicylate, homo-menthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di-2-ethylhexyl 4-methoxybenzmalonate;
triazine derivatives, such as, for example, 2,4,6-trianilino(p-carbo-2′-ethyl-1′-hexyloxy)-1,3,5-triazine and 2,4,6-tris[p-(2-ethylhexyloxy-carbonyl)anilino]-1,3,5-triazine (Uvinul T 150) as described in EP 0818450 A1 or bis(2-ethylhexyl) 4,4′-[(6-[4-((1,1-dimethylethyl)aminocarbonyl)phenylamino]-1,3,5-triazine-2,4-diyl)diimino]bis-benzoate (Uvasorb® HEB);
2,2-(methylenebis(6-(2H-benzotriazol-2-yl)-4-(1,1,3,3-tetramethylbutyl)phenol) (Tinosorb M);
2,4-bis[4-(2-ethylhexyloxy)-2-hydroxyphenyl]-6-(4-methoxyphenyl)-1,3,5-triazine (Tinosorb S);
propane-1,3-diones, such as, for example, 1-(4-tert-butylphenyl)-3-(4′-methoxyphenyl)propane-1,3-dione;
ketotricyclo(5.2.1.0)decane derivatives, as described in EP 0694521 B1;
dimethicodiethyl benzalmalonates (Parsol SLX).

Suitable water-soluble UV filters are:
2-phenylbenzimidazole-5-sulfonic acid and the alkali metal, alkaline earth metal, ammonium, alkylammonium, alkanolammonium and glucammonium salts thereof;
2,2-((1,4-phenylene)bis(1H-benzimidazole-4,6-disulfonic acid, monosodium salt) (Neo Heliopan AP)
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and salts thereof.

Suitable typical UV-A filters are in particular derivatives of benzoylmethane, such as, for example, 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF) and benzoic acid, 2-[4-(diethylamino)-2-hydroxybenzoyl], hexyl ester (Uvinul® A plus). The UV-A and UV-B filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Combinations of this type are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Suitable UV photoprotective filters are in particular the substances approved according to Annex VII of the Commission Directive (in the version Commission Directive 2005/9/EC of 28 Jan. 2005 amending Council Directive 76/768/EEC, concerning cosmetic products, for the purposes of adapting Annexes VII thereof to technical progress), to which reference is hereby explicitly made.

Besides the specified soluble substances, insoluble photoprotective pigments are also suitable for this purpose, namely finely disperse metal oxides and salts.

Examples of suitable metal oxides are in particular zinc oxide and titanium dioxide and also oxides of iron, zirconium, silicon, manganese, aluminum and cerium, and mixtures thereof. Salts which can be used are silicates (talc), barium sulfate or zinc stearate. The oxides and salts are used in the form of the pigments for skin care and skin-protecting emulsions and also for decorative cosmetics. The particles should have an average diameter of less than 100 nm, preferably between 5 and 50 nm and in particular between 15 and 30 nm. They can have a spherical shape, although it is also possible to use those particles which have an ellipsoidal shape or a shape which deviates in some other way from the spherical configuration. The pigments can also be present in surface-treated, i.e. hydrophilized or hydrophobicized, form. Typical examples thereof are coated titanium dioxides, such as, for example, Titanium dioxide T 805 (Degussa) or Eusolex® T, Eusolex® T-2000, Eusolex® T-Aqua, Eusolex® AVO, Eusolex® T-ECO, Eusolex® T-OLEO and Eusolex® T-S (Merck). Typical examples are zinc oxides, such as, for example, Zinc Oxide neutral, Zinc Oxide NDM (Symrise) or Z-Cote® (BASF) or SUNZnO-AS and SUNZnO-NAS (Sunjun Chemical Co. Ltd.). Suitable hydrophobic coatings here are primarily silicones and specifically trialkoxyoctylsilanes or simethicone. In sunscreen compositions, preference is given to using so-called micropigments or nanopigments. Preference is given to using micronized zinc oxide. Further suitable UV photoprotective filters can be found in the review by P. Finkel in SÖFW Journal 122, 8/1996, pp. 543-548 and Parf. Kosm. 80^{th} volume, No. 3/1999, p. 10 to 16.

Besides the two aforementioned groups of primary photoprotective substances, it is also possible to use secondary photoprotective agents of the antioxidant type, which interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. -carotene, -carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to mol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaicic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) suitable according to the invention of these specified active ingredients.

One subject matter of the invention therefore relates to cosmetic and/or pharmaceutical preparations comprising n-octyl n-octanoate and at least one UV photoprotective filter selected from the group consisting of 4-methylbenzylidene camphor, benzophenone-3, butyl methoxydibenzoylmethane, bis-ethyl-hexyloxyphenol methoxyphenyl triazine, methylene bis-benzotriazolyl tetramethylbutylphenol, diethylhexyl butamido triazone, ethylhexyl triazone and diethylamino hydroxybenzoyl hexyl benzoate, 3-(4′-trimethylammonium)benzylidenebornan-2-one methyl sulfate, 3,3′-(1,4-phenylenedimethine)bis(7,7-dimethyl-2-oxobicyclo-[2.2.1]heptane-1-methanesulfonic acid) and its salts, 3-(4′-sulfo)benzylidenebornan-2-one and its salts, polymer of N-{(2 and 4)-[2-oxoborn-3-ylidene]methyl}-benzyl]acrylamide, 2-(2H-benzotriazol-2-yl)-4-methyl-6-(2-methyl-3-(1,3,3,3-tetramethyl-1-(trimethylsilyloxy)disiloxanyl)propyl)phenol, dimethicodiethyl benzalmalonates and their mixtures. One subject matter of the invention therefore relates to cosmetic and/or pharmaceutical preparations comprising 0.1 to 80% by weight of n-octyl n-octanoate and at least one UV photoprotective filter selected from the group consisting of 4-methylbenzylidene camphor, benzophenone-3, butyl methoxydibenzoylmethane, bis-ethyl-hexyloxyphenol methoxyphenyl triazine, methylene bis-benzotriazolyl tetramethylbutylphenol, diethylhexyl butamido triazone, ethylhexyl triazone and diethylamino hydroxybenzoyl hexyl benzoate, 3-(4′-trimethylammonium)benzylidenebornan-2-one methyl sulfate, 3,3′-(1,4-phenylenedimethine)bis(7,7-dimethyl-2-oxobicyclo-[2.2.1]heptane-1-methanesulfonic acid) and its salts, 3-(4′-sulfo)benzylidenebornan-2-one and its salts, polymer of N-{(2 and 4)-[2-oxoborn-3-ylidene]methyl}-benzyl]acrylamide, 2-(2H-benzotriazol-2-yl)-4-methyl-6-(2-methyl-3-(1,3,3,3-tetramethyl-1-(trimethylsilyloxy)disiloxanyl)propyl)phenol, dimethicodiethyl benzalmalonates and their mixtures.

These UV photoprotective filters are commercially available, for example, under the following trade names:

NeoHeliopan®MBC (INCI: 4-Methylbenzylidene Camphor; manufacturer: Symrise); NeoHeliopan® BB (INCI: Benzophenone-3, manufacturer: Symrise); Parsol®1789 (INCI: Butyl Methoxydibenzoylmethane, manufacturer: Hoffmann-La Roche (Givaudan)); Tinosorb®S (INCI: Bis-Ethylhexyloxyphenol Methoxyphenyl Triazine); Tinosorb®M (INCI: Methylene Bis-Benzotriazolyl Tetramethylbutylphenol); manufacturer: Ciba Specialty Chemicals Corporation; Uvasorb®HEB (INCI: Diethylhexyl Butamido Triazone, manufacturer: 3V Inc.), Uvinul®T 150 (INCI: Ethylhexyl Triazone, manufacturer: BASF AG); Uvinul® A plus (INCI: Diethylamino Hydroxybenzoyl Hexyl Benzoate: manufacturer: BASF AG; Mexoryl® SO: 3-(4′-trimethylammonium)benzylidenebornan-2-one methyl sulfate, INCI: Camphor Benzalkonium Methosulfate; Mexoryl®SX: 3,3′-(1,4-phenylenedimethine)bis(7,7-dimethyl-2-oxobicyclo-[2.2.1]heptane-1-methanesulfonic acid), CTFA: INCI Terephthalylidene Dicamphor Sulfonic Acid; Mexoryl® SL: 3-(4′-sulfo)benzylidenebornan-2-one, INCI Benzylidene Camphor Sulfonic Acid; Mexoryl®SW: polymer of N-{(2 and 4)-[2-oxoborn-3-ylidene)methyl}benzyl]acrylamide, INCI Polyacrylamidomethyl Benzylidene Camphor; Mexoryl®SL: 2-(2H-benzotriazol-2-yl)-4-methyl-6-(2-methyl-3-(1,3,3,3-tetramethyl-1-(trimethylsilyloxy)disiloxanyl)propyl)phenol; INCI: DROMETRIZOLE TRISILOXANE; Parsol® SLX: dimethicodiethyl benzalmalonate, INCI Polysilicone-15.

The preparations according to the invention can comprise the UV photoprotective filters in amounts of from 0.5 to 30% by weight, preferably 2.5 to 20% by weight, particularly preferably 5-15% by weight—based on the total weight of the cosmetic and/or pharmaceutical preparation.

One subject matter of the invention relates to cosmetic and/or pharmaceutical preparations comprising n-octyl n-octanoate and at least one self-tanning agent. A preferred subject matter of the invention relates to cosmetic and/or pharmaceutical preparations comprising 0.1 to 80% by weight of n-octyl n-octanoate and at least one self-tanning agent.

Self-tanning agents are to be understood as meaning substances which cause tanning of the skin. By way of example, mention may be made of alpha,beta-unsaturated aldehydes which react with the amino acids in the skin in the sense of a Maillard reaction to give colored compounds. Further suitable active ingredients for self-tanning agents are natural or synthetic ketols and aldols. Suitable active ingredients which may be mentioned by way of example are dihydroxyacetone, erythrulose glycerol aldehyde, alloxan, hydroxymethyl-glyoxal, gamma-dialdehyde, 6-aldo-D-fructose, ninhydrin and meso-tartardialdehyde. Suitable self-tanning agents are in particular dihydroxyacetone and/or erythrulose. Mixtures of the aforementioned active ingredients with one another or with mucondialdehyde and/or naphthoquinones such as, for example, 5-hydroxy-1,4-naphthoquinone (juglone) and 2-hydroxy-1,4-naphthoquinone have proven to be particularly advantageous. The compositions according to the invention comprise the self-tanning agents usually in concentrations of from 1 to 10, in particular from 2 to 5% by weight—based on the total weight of the cosmetic and/or pharmaceutical preparation.

One subject matter of the invention relates to cosmetic and/or pharmaceutical preparations comprising n-octyl n-octanoate and at least one self-tanning agent and at least one UV photoprotective filter. A preferred subject matter of the invention relates to cosmetic and/or pharmaceutical preparations comprising 0.1 to 80% by weight of n-octyl n-octanoate and at least one UV photoprotective filter and at least one self-tanning agent.

The cosmetic and/or pharmaceutical preparations according to the invention may be present, for example, as O/W or W/O care emulsions, sunscreen formulation, antiperspirant/deodorant concepts, formulations for decorative cosmetics, oily care preparations, impregnation fluids for substrates, such as, for example, paper and nonwoven products. By way of example, mention may be made of wet wipes, pocket tissues, diapers or hygiene products.

The n-octyl n-octanoate according to the invention and the cosmetic and/or pharmaceutical preparations according to the invention are suitable in particular also for light, sprayable applications and/or as constituents of care emulsions for tissues, papers, wipes, sponges (e.g. polyurethane sponges), plasters in the sector of baby hygiene, baby care, skincare, sun protection, aftersun treatment, insect repellent, cleansing, facial cleansing and antiperspirant/deodorant application. They can be applied to tissues, papers, wipes, nonwoven products, sponges, puffs, plasters and bandages which are used in the field of cleansing, hygiene and/or care (wet wipes for baby hygiene and baby care, cleansing wipes, facial cleansing wipes, skincare wipes, care wipes with active ingredients to combat skin aging, wipes with sunscreen formulations and insect repellents and also wipes for decorative cosmetics or for aftersun treatment, toilet wet wipes, antiperspirant wipes, diapers, tissues, wet wipes, hygiene products, self-tanning wipes, toilet paper, refreshing wipes, aftershave wipes). They can be used inter alia also in preparations for haircare, hair cleaning or hair coloring. The use of the n-octyl n-octanoate according to the invention has a positive influence on the sensory behavior upon application.

The n-octyl n-octanoate according to the invention is suitable in particular as constituent of decorative cosmetic preparations, such as, for example, lip pencils, eye make-up, such as, for example, eye shadows, mascara, eyeliners, kohl, nail varnish, etc., and also make-up formulations. One subject matter of the invention relates to cosmetic and/or pharmaceutical preparations comprising n-octyl n-octanoate and at least one pigment and/or a dye. A preferred subject matter of the invention relates to cosmetic and/or pharmaceutical preparations comprising 0.1 to 80% by weight of n-octyl n-octanoate and at least one pigment and/or a dye.

The term pigment covers particles of any shape, which are white or colored, organic or inorganic, are insoluble in the preparations, and serve the purpose of coloring the preparation. In one preferred embodiment, inorganic pigments are used, particular preference being given to metal oxides.

Examples of inorganic pigments which may be mentioned are: titanium dioxide, optionally surface-coated, zirconium or cerium oxides and zinc, iron (black, yellow or red) and chromium oxides, manganese violet, ultramarine blue, chromium hydrate and iron(III) blue, metal powder such as aluminum powder or copper powder. In a preferred embodiment of the invention, the pigment is selected from the inorganic pigments, preferably from the metal oxides. In a preferred embodiment, the pigment is selected from the group consisting of titanium dioxide, zinc oxide, iron oxide and mixtures thereof.

The pigments can be present either individually or in mixtures. Within the context of the present invention, preference is given to pigment mixtures composed of white pigments (e.g. kaolin, titanium dioxide or zinc oxide) and inorganic colored pigments (e.g. iron oxide pigments, chromium oxides), where the pigments may be present in coated or uncoated form. Among the colored pigments, iron oxides are particularly preferred. Within the context of the present invention, the pigment(s) may also advantageously be selected from the group of the effect pigments, which impart to the cosmetic preparation, as well as the pure color, an additional property—such as e.g. angular dependence of the color (flop), luster (not surface luster) or texture. According to the invention, such effect pigments are used advantageously in addition to one or more white pigments and/or colored pigments.

The most important group of effect pigments is that of the luster pigments, which, according to DIN 55944: 2003-11, include the metal effect pigments and the pearlescent pigments. Some specific effect pigments cannot be assigned to these two groups, e.g. graphite platelets, iron oxide platelets and micronized titanium dioxide, where micronized titanium dioxide does not give a luster effect, but an angle-dependent light-scattering effect. The luster pigments according to DIN 55943: 2001-10 are predominantly effect pigment platelets. Oriented in parallel, luster pigments exhibit a characteristic luster. The visual effect of luster pigments is based on the directed reflection on metallic particles (metal effect pigments), on transparent particles with a high refractive index (pearlescent pigments) or on the phenomenon of interference (interference pigments) (DIN 55944: 2003-11). Examples of commercially available effect pigments preferred according to the invention are: Timiron and #174; from Merck, Iriodin and #174; from Merck (pearlescent and color luster pigments for decorative technical applications), Xirallic and #174; from Merck (color-intense crystal effect pigments).

In addition, the preparations according to the invention can also advantageously comprise organic colored pigments, i.e. organic dyes which are virtually insoluble in the preparation. According to DIN 55944: 1990-04, organic pigments can be divided according to chemical aspects into azo pigments and polycyclic pigments, and according to color aspects into chromatic or black pigments. Organic white pigments are of no practical significance.

Within the context of the present invention, the pigments may advantageously also be used in the form of commercially available oily or aqueous predispersions. The preparations according to the invention usually comprise 0.1 to 40% by weight of pigments—based on the total weight of the cosmetic and/or pharmaceutical preparation.

It is also advantageous within the context of the present invention if the preparation according to the invention comprises one or more dyes. The dyes may be either of synthetic or natural origin. A list of suitable dyes can be found in EP 1 371 359 A2, p. 8, 1. 25-57, p. 9 and p. 10, and also p. 11, 1. 1 to 54, to which reference is hereby explicitly made. The preparations according to the invention usually comprise 0.01 to 5, preferably 0.1 to 1.0% by weight, of dyes—based on the total weight of the cosmetic and/or pharmaceutical preparation. The preparations according to the invention usually comprise a total amount of dyes and pigments in the range from 0.01 to 30% by weight, in particular 0.1 to 15% by weight, preferably 1 to 10% by weight, based on the total weight of the cosmetic and/or pharmaceutical preparation.

Suitable dyes and pigments are in particular the dyes and pigments approved according to Annex IV of the Commission Directive (in the version: Commission Directive 2007/22/EC of 17 Apr. 2007 amending Council Directive 76/768/EEC, concerning cosmetic products, for the purposes of adapting Annexes IV and VI thereto to technical progress) approved substances, to which reference is hereby explicitly made.

The cosmetic and/or pharmaceutical preparations may be formulations for body care, e.g. a body milk, creams, lotions, sprayable emulsions, products for eliminating body odor etc. The n-octyl n-octanoate can also be used in surfactant-containing formulations, such as e.g. foam baths and shower baths, hair shampoos and care rinses. Depending on the intended application, the cosmetic and/or pharmaceutical preparations comprise a series of further auxiliaries and additives, such as, for example, surfactants, further oil bodies, emulsifiers, pearlescent waxes, consistency regulators, thickeners, superfatting agents, stabilizers, polymers, fats, waxes, lecithins, phospholipids, biogenic active ingredients, antidandruff agents, film formers, swelling agents, insect repellents, self-tanning agents, tyrosinase inhibitors (depigmenting agents), hydrotropes, solubilizers, preservatives, perfume oils, dyes etc., which are listed below by way of example.

A subject matter of the invention relates to cosmetic and/or pharmaceutical preparations comprising n-octyl n-octanoate and at least one emulsifier and/or a surfactant and/or a wax component and/or a polymer and/or a further oil body. A preferred subject matter of the invention relates to cosmetic and/or pharmaceutical preparations comprising 0.1 to 80% by weight of n-octyl n-octanoate and at least one emulsifier and/or a surfactant and/or a wax component and/or a polymer and/or a further oil body.

Emulsifier

In one embodiment of the invention, the preparations according to the invention comprise at least one emulsifier. One subject matter of the invention relates to cosmetic and/or pharmaceutical preparations comprising n-octyl n-octanoate and at least one emulsifier. A preferred subject matter of the invention relates to cosmetic and/or pharmaceutical preparations comprising 0.1 to 80% by weight of n-octyl n-octanoate and at least one emulsifier. The compositions according to the invention comprise the emulsifier(s) usually in an amount of from 0 to 40% by weight, preferably 0.1 to 20% by weight, preferably 0.1 to 15% by weight and in particular 0.1 to 10% by weight, based on the total weight of the composition.

Every emulsifier is assigned a so-called HLB value (a dimensionless number between 0 and 20) which indicates whether there is a preference for water or oil solubility. Numbers below 9 indicate preferentially oil-soluble, hydrophobic emulsifiers; numbers above 11 water-soluble, hydrophilic emulsifiers. The HLB value says something about the equilibrium of the size and strength of the hydrophilic and lipophilic groups of an emulsifier. The HLB value of an emulsifier can also be calculated from increments, where the HLB increments for the different hydrophilic and hydrophobic groups from which a molecule is composed. As a rule, it can be found in tabular works (e.g. H. P. Fiedler, Lexikon der Hilfsstoffe fur Pharmazie, Kosmetik and angrenzende Gebiete [Lexicon of the auxiliaries for pharmacy, cosmetics and related fields], Editio Cantor Verlag, Aulendorf, 4th Edition 1996) or manufacturer data. The solubility of the emulsifier in the two phases in practice determines the emulsion type. If the emulsifier has better solubility in water, an O/W emulsion is obtained. If, on the other hand, the emulsifier has better solubility in the oil phase, a W/O emulsion arises under otherwise identical production conditions.

In one embodiment of the invention, the preparation according to the invention comprises more than one emulsifier. Depending on the other components, the person skilled in the art uses customary emulsifier systems (such as e.g. emulsifier and coemulsifier).

Nonionic Emulsifiers

The group of nonionic emulsifiers includes, for example:
(1) Addition products of from 2 to 50 mol of ethylene oxide and/or from 1 to 20 mol of propylene oxide onto linear fatty alcohols having 8 to 40 carbon atoms, onto fatty acids having 12 to 40 carbon atoms and onto alkylphenols having 8 to 15 carbon atoms in the alkyl group.
(2) C₁₂-C₁₈ fatty acid mono- and diesters of addition products of from 1 to 50 mol of ethylene oxide onto glycerol.
(3) Sorbitan mono- and diesters of saturated and unsaturated fatty acids having 6 to 22 carbon atoms and ethylene oxide addition products thereof.
(4) Alkyl mono- and oligoglycosides having 8 to 22 carbon atoms in the alkyl radical and ethoxylated analogs thereof.
(5) Addition products of from 7 to 60 mol of ethylene oxide onto castor oil and/or hydrogenated castor oil.
(6) Polyol and in particular polyglycerol esters, such as, for example, polyol poly-12-hydroxystearates, polyglycerol polyricinoleate, polyglycerol diisostearate or polyglycerol dimerate. Likewise suitable are mixtures of compounds of two or more of these substance classes.
(7) Addition products of from 2 to 15 mol of ethylene oxide onto castor oil and/or hydrogenated castor oil.
(8) Partial esters based on linear, branched, unsaturated or saturated C₆-C₂₂-fatty acids, ricinoleic acid and 12-hydroxystearic acid and polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose), or mixed esters such as, for example, glyceryl stearate citrate and glyceryl stearate lactate.
(9) Polysiloxane-polyalkyl-polyether copolymers and corresponding derivatives.
(10) Mixed esters of pentaerythritol, fatty acids, citric acid and fatty alcohol and/or mixed esters of fatty acids having 6 to 22 carbon atoms, methylglucose and polyols, preferably glycerol or polyglycerol.

The addition products of ethylene oxide and/or of propylene oxide onto fatty alcohols, fatty acids, alkylphenols, glycerol mono- and diesters and also sorbitan mono- and diesters of fatty acids and onto castor oil are known, commercially available products. These are homolog mixtures whose average degree of alkoxylation corresponds to the ratio of the quantitative amounts of ethylene oxide and/or propylene oxide and substrate with which the addition reaction is carried out. Depending on the degree of ethoxylation, they are W/O or O/W emulsifiers. C_{12/18}-fatty acid mono- and diesters of addition products of ethylene oxide onto glycerol are known as refatting agents for cosmetic preparations.

Particularly highly suitable and mild emulsifiers according to the invention are polyol poly-12-hydroxystearates and mixtures thereof, which are sold, for example, under the trade names “Dehymuls® PGPH” (W/O emulsifier) or “Eumulgin® VL 75” (mixture with Lauryl Glucosides in the weight ratio 1:1, O/W emulsifier) or Dehymuls® SBL (W/O emulsifier) by Cognis Deutschland GmbH. In this connection, reference may be made in particular to the European patent EP 766 661 B1. The polyol component of these emulsifiers can be derived from substances which have at least two, preferably 3 to 12 and in particular 3 to 8, hydroxyl groups and 2 to 12 carbon atoms. Particularly preferred emulsifiers are, for example, Cetyl Dimethicone Copolyol (e.g. Abil EM-90), Polyglyceryl-2 Dipolyhydroxystearate (e.g. Dehymuls PGPH), Polyglycerin-3-Diisostearate (e.g. Lameform TGI), Polyglyceryl-4 Isostearate (e.g. Isolan GI 34), Polyglyceryl-3 Oleate (e.g. Isolan GO 33), Diisostearoyl Polyglyceryl-3 Diisostearate (e.g. Isolan PDI), Polyglyceryl-3 Methylglucose Distearate (e.g. Tego Care 450), Polyglyceryl-3 Beeswax (e.g. Cera Bellina), Polyglyceryl-4 Caprate (e.g. Polyglycerol Caprate T2010/90), Polyglyceryl-3 Cetyl Ether (e.g. Chimexane NL), Polyglyceryl-3 Distearate (e.g. Cremophor GS 32) and Polyglyceryl Polyricinoleate (e.g. Admul WOL 1403), Glyceryl Oleate (e.g. Monomuls 90-O 18), Alkyl Glucoside (e.g. Plantacare 1200, Emulgade PL 68/50, Montanov 68, Tego Care CG 90, Tego Glucosid L 55), Methyl Glucose Isostearate (e.g. Tego Care IS), Methyl Glucose Sesquistearate (Tego Care PS), Sodium Cocoyl Hydrolyzed Wheat Protein (e.g. Gluadin WK), Potassium Cetyl Phosphate (e.g. Amphisol K, Crodafos CKP), Sodium Alkylsulfate (e.g. Lanette E), Glyceryl Stearate Citrate (e.g. Axol C 62, Dracorin CE 614035, Imwitor 372P and 370), Sucrose Ester (e.g. Crodesta F-10, F-20, F-50, F-70, F-110, F-160, SL-40, Emulgade® Sucro), ethoxylated and/or propoxylated fatty alcohols fatty acids, castor oils and hydrogenated castor oils (e.g. Eumulgin B2, B2, B3, L, HRE 40, HRE 60, RO 40, Cremophor HRE 40, HRE 60, L, WO 7, Dehymuls HRE 7, Arlacel 989), PEG-30 Dipolyhydroxystearate (e.g. Arlacel P 135, Dehymuls LE), sorbitan esters, sorbitan esters ethoxylated and/or propoxylated and mixtures thereof. A particularly effective mixture consists of Polyglyceryl-2 Dipolyhydroxystearate and Lauryl Glucoside and Glycerol (e.g. Eumulgin VL 75). Also suitable are Polyglyceryl-4 Diisostearate/Polyhydroxystearate/Sebacate (Isolan® GPS), Diisostearoyl Polyglyceryl-3 Diisostearate (e.g. Isolan PDI), alkali metal salts Acylglutamate (e.g. Eumulgin SG).

Of suitability in principle as lipophilic W/O emulsifiers are emulsifiers with an HLB value of from 1 to 8 which are summarized in numerous tables and are known to the person skilled in the art. Some of these emulsifiers are listed, for example, in Kirk-Othmer, “Encyclopedia of Chemical Technology”, 3^{rd} edition, 1979, volume 8, page 913. For ethoxylated products, the HLB value can also be calculated according to the following formula: HLB=(100−L):5, where L is the weight fraction of the lipophilic groups, i.e. of the fatty alkyl or fatty acyl groups in weight percent, in the ethylene oxide adducts.

Of particular advantage from the group of W/O emulsifiers are partial esters of polyols, in particular of C₄-C₆-polyols, such as, for example, partial esters of pentaerythritol or sugar esters, e.g. sucrose distearate, sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical-grade mixtures thereof. Also suitable as emulsifiers are addition products of from 1 to 30, preferably 5 to 10, mol of ethylene oxide onto the specified sorbitan esters.

Depending on the formulation, it may be advantageous to additionally use at least one emulsifier from the group of nonionic O/W emulsifiers (HLB value: 8-18) and/or solubilizers. These are, for example, the ethylene oxide adducts already mentioned in the introduction and having a correspondingly high degree of ethoxylation, e.g. 10-20 ethylene oxide units for O/W emulsifiers and 20-40 ethylene oxide units for so-called solubilizers. According to the invention, Ceteareth-12 and PEG-20 Stearate are particularly advantageous as O/W emulsifiers. Suitable solubilizers are preferably Eumulgin® HRE 40 (INCI: PEG-40 Hydrogenated Castor Oil), Eumulgin® HRE 60 (INCI: PEG-60 Hydrogenated Castor Oil), Eumulgin® L (INCI: PPG-1-PEG-9 Lauryl Glycol Ether), and Eumulgin® SML 20 (INCI: Polysorbate-20).

Nonionic emulsifiers from the group of alkyl oligoglycosides are particularly skin-friendly and therefore preferably suitable as O/W emulsifiers. C₈-C₂₂-alkyl mono and oligoglycosides, their preparation and their use are known from the prior art. Their preparation takes place in particular by reacting glucose or oligosaccharides with primary alcohols having 8 to 22 carbon atoms. As regards the glycoside radical, either monoglycosides, in which a cyclic sugar radical is glycosidically bonded to the fatty alcohol, or oligomeric glycosides with a degree of oligomerization up to preferably about 8 are suitable. The degree of oligomerization here is a statistical average value based on a homolog distribution customary for such technical-grade products. Products which are available under the name Plantacare® comprise a glucosidically bonded C₈-C₁₆-alkyl group onto an oligoglucoside radical whose average degree of oligomerization is 1 to 2. The acylglucamides derived from glucamine are also suitable as nonionic emulsifiers. According to the invention, preference is given to a product which is sold under the name Emulgade® PL 68/50 by Cognis Deutschland GmbH and is a 1:1 mixture of alkyl polyglucosides and fatty alcohols. According to the invention, it is also advantageously possible to use a mixture of Lauryl Glucoside, Polyglyceryl-2 Dipolyhydroxystearate, glycerol and water, which is commercially available under the name Eumulgin® VL 75.

Also suitable as emulsifiers are substances such as lecithins and phospholipids. Examples of natural lecithins which may be mentioned are the cephalins, which are also referred to as phosphatidic acids and are derivatives of 1,2-diacyl-sn-glycerol-3-phosphoric acids. By contrast, phospholipids are usually understood as meaning mono- and preferably diesters of phosphoric acid with glycerol (glycerol phosphates), which are generally included in the fats. In addition, sphingosines and sphingolipids are also suitable. Silicone emulsifiers, for example, may be present as emulsifiers. These can be selected, for example, from the group of alkylmethicone copolyols and/or alkyldimethicone copolyols, in particular from the group of compounds which are characterized by the following chemical structure:

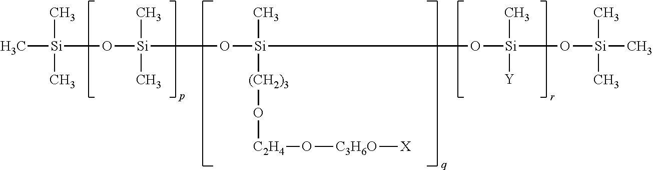

in which X and Y, independently of one another, are selected from the group H (hydrogen) and the branched and unbranched alkyl groups, acyl groups and alkoxy groups having 1-24 carbon atoms, p is a number from 0-200, q is a number from 1-40, and r is a number from 1-100.

One example of silicone emulsifiers to be used particularly advantageously within the context of the present invention are dimethicone copolyols, which are sold by Evonik Goldschmidt under the trade names AXIL® B 8842, ABIL® B 8843, ABIL® B 8847, ABIL® B 8851, ABIL® B 8852, ABIL® B 8863, ABIL® B 8873 and ABIL® B 88183. A further example of interface-active substances to be used particularly advantageously within the context of the present invention is cetyl PEG/PPG-10/1 dimethicone (Cetyl Dimethiconecopolyol), which is sold by Evonik Goldschmidt under the trade name ABIL® EM 90. A further example of interface-active substances to be used particularly advantageously within the context of the present invention is the cyclomethicone dimethiconecopolyol, which is sold by Evonik Goldschmidt under the trade name ABIL®EM 97 and ABIL®WE 09. Furthermore, the emulsifier Lauryl PEG/PPG-18/18 Methicone (laurylmethicone copolyol) has proven to be very particularly advantageous and is available under the trade name Dow Corning® 5200 Formulation Aid from Dow Corning Ltd. A further advantageous silicone emulsifier is Octyl Dimethicone Ethoxy Glucoside from Wacker. For a water-in-silicone oil emulsion according to the invention, all known emulsifiers used for this type of emulsion can be used. According to the invention, particularly preferred water-in-silicone emulsifiers here are cetyl PEG/PPG-10/1 dimethicone and lauryl PEG/PPG-18/18 methicone [e.g. ABIL® EM 90 (Evonik Goldschmidt), DC5200 Formulation Aid (Dow Corning)] and any desired mixtures of the two emulsifiers.

Surfactants

In one embodiment of the invention, the preparations according to the invention comprise at least one surfactant. Surfactants are amphiphilic substances which can dissolve organic, nonpolar substances in water. As a result of their specific molecular structure with at least one hydrophilic and one hydrophobic molecular moiety, they provide for a reduction in the surface tension of water, wetting of the skin, facilitation of soil removal and release, ease of rinsing off and—if desired for foam regulation. Surfactants are usually understood as meaning surface-active substances which have an HLB value greater than 20.

One subject matter of the invention relates to cosmetic and/or pharmaceutical preparations comprising n-octyl n-octanoate and at least one surfactant. A preferred subject matter of the invention relates to cosmetic and/or pharmaceutical preparations comprising 0.1 to 80% by weight of n-octyl n-octanoate and at least one surfactant.

Surface-active substances which may be present are anionic, nonionic, cationic and/or amphoteric or zwitterionic surfactants. In surfactant-containing cosmetic preparations, such as, for example, shower gels, foam baths, shampoos etc., at least one anionic surfactant is preferably present. The compositions according to the invention comprise the surfactant(s) usually in an amount of from 0 to 40% by weight, preferably 0.05 to 30% by weight, in particular 0.05 to 20% by weight, preferably 0.1 to 15% by weight and in particular 0.1 to 10% by weight, based on the total weight of the composition.

Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partially oxidized alk(en)yl oligoglycosides and glucuronic acid derivatives, fatty acid N-alkylglucamides, protein hydrolyzates (in particular wheat-based vegetable products), polyol fatty acid esters, sugar esters, sorbitan esters, polysorbates and amine oxides. If the nonionic surfactants contain polyglycol ether chains, these may have a conventional homolog distribution, but preferably have a narrowed homolog distribution.

Zwitterionic surfactants is the term used to refer to those surface-active compounds which carry at least one quaternary ammonium group and at least one —COO⁽⁻⁾ or —SO₃⁽⁻⁾ group in the molecule. Particularly suitable zwitterionic surfactants are the so-called betaines, such as the N-alkyl-N,N-dimethylammonium glycinates, for example cocoalkyl dimethylammonium glycinate,

N-acylaminopropyl-N,N-dimethylammonium glycinates, for example cocoacylaminopropyldimethylammonium glycinate, and 2-alkyl-3-carboxymethyl-3-hydroxyethylimidazoline having in each case 8 to 18 carbon atoms in the alkyl or acyl group, and also cocoacylaminoethyl hydroxyethylcarboxymethyl glycinate. A preferred zwitterionic surfactant is the fatty acid amide derivative known under the INCI name Cocamidopropyl Betaine.

Likewise suitable, especially as cosurfactants, are ampholytic surfactants. Ampholytic surfactants are understood as meaning those surface-active compounds which, apart from a C₈-C₁₈-alkyl or acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkylimino-dipropionic acids, N-hydroxyethyl-N-alkylamidopropyl-glycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethyl-aminopropionate and C₁₂₋₁₈-acylsarcosine. Typical examples of amphoteric and zwitterionic surfactants are alkylbetaines, alkylamidobetaines, aminopropionates, aminoglycinates, imidazolinium betaines and sulfo-betaines. The specified surfactants are exclusively known compounds. With regard to the structure and preparation of these substances, reference may be made to relevant review works in this field. Typical examples of particularly suitable mild, i.e. particularly skin-friendly, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides and/or mixtures thereof with alkyl oligoglucoside carboxylates, fatty acid glucamides, alkylamidobetaines, amphoacetals and/or protein fatty acid condensates, the latter preferably based on wheat proteins or salts thereof. Anionic surfactants are characterized by a water-solubilizing, anionic group, such as, for example, a carboxylate, sulfate, sulfonate, citrate, or phosphate group and a lipophilic radical. Skin-compatible anionic surfactants are known to the person skilled in the art in a large number from relevant handbooks and are commercially available. These are in particular alkyl sulfates in the form of their alkali metal, ammonium or alkanolammonium salts, alkyl ether sulfates, alkyl ether carboxylates, acyl isethionates, acyl sarcosinates, acyl taurines with linear alkyl or acyl groups having 12 to 18 carbon atoms, and also sulfosuccinates and acyl glutamates in the form of their alkali metal or ammonium salts. Typical examples of anionic surfactants are soaps, alkylbenzenesulfonates, alkanesulfonates, olefinsulfonates, alkyl ether sulfonates, glycerol ether sulfonates, α-methyl ester sulfonates, sulfo fatty acids, alkyl sulfates, glyceryl stearate citrate, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ethercarboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, protein fatty acid condensates (in particular vegetable products based on wheat) and alkyl (ether) phosphates. If the anionic surfactants comprise polyglycol ether chains, these may have a conventional homolog distribution, but preferably have a narrowed homolog distribution. Cationic surfactants which can be used are in particular quaternary ammonium compounds. Preference is given to ammonium halides, in particular chlorides and bromides, such as alkyltrimethylammonium chlorides, dialkyldimethylammonium chlorides and trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Furthermore, the very readily biodegradable quaternary ester compounds, such as, for example, the dialkylammonium methosulfates and methylhydroxyalkyldialkoyl-oxyalkylammonium methosulfates sold under the trade name Stepantex® and the corresponding products of the Dehyquart® series can also be used as cationic surfactants. The term “esterquats” is generally understood as meaning quaternized fatty acid triethanolamine ester salts. They can impart a particular soft feel to the preparations according to the invention. These are known substances which are prepared by the relevant methods of organic chemistry. Further cationic surfactants which can be used according to the invention are the quaternized protein hydrolyzates.

Wax Component

In one embodiment of the invention, the preparations according to the invention comprise at least one wax component. One subject matter of the invention relates to cosmetic and/or pharmaceutical preparations comprising n-octyl n-octanoate and at least one wax component. One preferred subject matter of the invention relates to cosmetic and/or pharmaceutical preparations comprising 0.1 to 80% by weight of n-octyl n-octanoate and at least one wax component. The compositions according to the invention comprise the wax component(s) usually in an amount of from 0 to 40% by weight, in particular from 0 to 20% by weight, preferably 0.1 to 15% by weight and in particular 0.1 to 10% by weight, based on the total weight of the composition.

The term wax is usually understood as meaning all natural or artificially obtained substances and substance mixtures having the following properties: they are from solid to brittly hard consistency, coarse to finely crystalline, transparent to cloudy and melt above 30° C. without decomposition. They are low viscosity even a little above the melting point and are not thread-drawing and exhibit a strongly temperature-dependent consistency and solubility. According to the invention, it is possible to use a wax component or a mixture of wax components which melt at 30° C. or above. Waxes which can be used according to the invention are also fats and fat-like substances with wax-like consistency provided they have the required melting point. These include, inter alia, fats (triglycerides), mono- and diglycerides, natural and synthetic waxes, fatty and wax alcohols, fatty acids, esters of fatty alcohols and fatty acids and also fatty acid amides or any desired mixtures of these substances. Fats are understood as meaning triacylglycerols, i.e. the triple esters of fatty acids with glycerol. Preferably, they comprise saturated, unbranched and unsubstituted fatty acid radicals. These may also be mixed esters, i.e. triple esters of glycerol with various fatty acids. According to the invention so-called hydrogenated fats and oils, which are obtained by partial hydrogenation, can be used and are particularly highly suited as consistency regulators. Vegetable hydrogenated fats and oils are preferred, e.g. hydrogenated castor oil, peanut oil, soybean oil, colza oil, rapeseed oil, cottonseed oil, soybean oil, sunflower oil, palm oil, palm kernel oil, linseed oil, almond oil, corn oil, olive oil, sesame oil, cocoa butter and coconut fat. Inter alia, the triple esters of glycerol with C12-C60-fatty acids and in particular C12-C36-fatty acids are suitable. These include hydrogenated castor oil, a triple ester of glycerol and a hydroxystearic acid, which is commercially available, for example, under the name Cutina HR. Glycerol tristearate, glycerol tribehenate (e.g. Syncrowax HRC), glycerol tripalmitate or the triglyceride mixtures known under the name Syncrowax HGLC are likewise suitable, with the proviso that the melting point of the wax component or of the mixture is 30° C. or above. According to the invention, wax components which can be used are in particular mono- and diglycerides and mixtures of these partial glycerides. Glyceride mixtures which can be used according to the invention include the products Novata AB and Novata B (mixture of C12-C18-mono-, di- and triglycerides) and Cutina MD or Cutina GMS (glyceryl stearate) marketed by Cognis Deutschland GmbH & Co. KG. Fatty alcohols which can be used according to the invention as wax component include the C12-C50-fatty alcohols. The fatty alcohols can be obtained from natural fats, oils and waxes, such as, for example, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. According to the invention, preference is given to saturated unbranched fatty alcohols. However, unsaturated, branched or unbranched fatty alcohols can also be used according to the invention as wax component provided they have the required melting point. According to the invention, it is also possible to use fatty alcohol cuts as are produced during the reduction of naturally occurring fats and oils, such as, for example, bovine tallow, peanut oil, colza oil, cottonseed oil, soybean oil, sunflower oil, palm kernel oil, linseed oil, castor oil, corn oil, rapeseed oil, sesame oil, cocoa butter and coconut fat. However, it is also possible to use synthetic alcohols, e.g. the linear, even-numbered fatty alcohols of the Ziegler synthesis (alfols) or the partially branched alcohols from the oxo synthesis (dobanols). According to the invention, C14-C22-fatty alcohols, which are marketed, for example, by Cognis Deutschland GmbH under the name Lanette 18 (C18-alcohol), Lanette 16 (C16-alcohol), Lanette 14 (C14-alcohol), Lanette O (C16/C18-alcohol) and Lanette 22 (C18/C22-alcohol), are particularly preferably suitable. Fatty alcohols give the compositions a dryer skin feel than triglycerides and are therefore preferred over the latter. Wax components which can be used are also C14-C40-fatty acids or mixtures thereof. These include, for example, myristic acid, pentadecanoic acid, palmitic acid, margaric acid, stearic acid, nonadecanoic acid, arachic acid, behenic acid, lignoceric acid, cerotic acid, melissic acid, erucic acid and elaeostearic acid, and also substituted fatty acids, such as, for example, 12-hydroxystearic acid, and the amides or monoethanolamides of the fatty acids, this list being exemplary and nonlimiting in character. According to the invention, it is possible to use, for example, natural vegetable waxes, such as candelilla wax, carnauba wax, Japan wax, esparto grass wax, cork wax, guaruma wax, rice germ oil wax, sugarcane wax, ouricury wax, montan wax, sunflower wax, fruit waxes such as orange waxes, lemon waxes, grapefruit wax, bayberry wax, and animal waxes, such as, for example, beeswax, shellac wax, spermaceti, wool wax and uropygial grease. Within the context of the invention, it may be advantageous to use hydrogenated or hardened waxes. Natural waxes that can be used according to the invention also include mineral waxes, such as, for example, ceresin and ozokerite or the petrochemical waxes, such as, for example, petrolatum, paraffin waxes and microwaxes. Wax components which can be used are also chemically modified waxes, in particular the hard waxes, such as, for example, montan ester waxes, sasol waxes and hydrogenated jojoba waxes. Synthetic waxes which can be used according to the invention include, for example, wax-like polyalkylene waxes and polyethylene glycol waxes. Vegetable waxes are preferred according to the invention.

The wax component can likewise be selected from the group of wax esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (e.g. 12-hydroxystearic acid) and saturated and/or unsaturated, branched and/or unbranched alcohols, and also from the group of lactides of long-chain hydroxycarboxylic acids. Examples of such esters are the C16-C40-alkyl stearates, C20-C40-alkyl stearates (e.g. Kesterwachs K82H), C20-C40-dialkyl esters of dimeric acids, C18-C38-alkylhydroxystearoyl stearates or C20-C40-alkyl erucates. C30-C50-Alkylbeeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate can also be used. Fatty acid partial glycerides, i.e. technical-grade mono- and/or diesters of glycerol with fatty acids having 12 to 18 carbon atoms, such as, for example, glycerol mono/dilaurate, -palmitate, -myristate or -stearate, are also suitable for this purpose. Suitable waxes are also pearlescent waxes. Suitable pearlescent waxes, especially for use in surface-active formulations, are, for example: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have in total at least 24 carbon atoms, specifically laurone and distearyl ethers; fatty acids such as stearic acid, hydroxystearic acid or behenic acid, ring-opening products of olefin epoxides having 12 to 22 carbon atoms with fatty alcohols having to 22 carbon atoms and/or polyols having 2 to 15 carbon atoms and 2 to 10 hydroxyl groups, and mixtures thereof.

Polymers

In one embodiment of the invention, the preparations according to the invention comprise at least one polymer. One subject matter of the invention relates to cosmetic and/or pharmaceutical preparations comprising n-octyl n-octanoate and at least one polymer. One preferred subject matter of the invention relates to cosmetic and/or pharmaceutical preparations comprising 0.1 to 80% by weight of n-octyl n-octanoate and at least one polymer. The compositions according to the invention comprise the polymer(s) usually in an amount of from 0 to 20% by weight, preferably 0.1 to 15% by weight and in particular 0.1 to 10% by weight, based on the total weight of the composition.

Suitable cationic polymers are, for example, cationic cellulose derivatives, such as, for example, a quaternized hydroxyethylcellulose, which is available under the name Polymer JR 400® from Amerchol, cationic starch, copolymers of diallylammonium salts and acrylamides, quaternized vinylpyrrolidone/vinylimidazole polymers, such as, for example, Luviquat® (BASF), condensation products of polyglycols and amines, quaternized collagen polypeptides, such as, for example, lauryldimonium hydroxypropyl hydrolyzed collagen (Lamequat®L/Grünau), quaternized wheat polypeptides, polyethylenimine, cationic silicone polymers, such as, for example, amidomethicones, copolymers of adipic acid and dimethylaminohydroxy-propyldiethylenetriamine (Cartaretine®/Sandoz), copolymers of acrylic acid with dimethyldiallylammonium chloride (Merquat® 550/Chemviron), polyaminopolyamides, cationic chitin derivatives such as, for example, quaternized chitosan, optionally in microcrystalline distribution, condensation products of dihaloalkylene, such as, for example, dibromobutane with bisdialkylamines, such as, for example, bisdimethylamino-1,3-propane, cationic guar gum, such as, for example, Jaguar® CBS, Jaguar® C-17, Jaguar® C-16 from Celanese, quaternized ammonium salt polymers, such as, for example, Mirapol® A-15, Mirapol® AD-1, Mirapol® AZ-1 from Miranol.

Suitable anionic, zwitterionic, amphoteric and nonionic polymers are, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyltrimethylammonium chloride/acrylate copolymers, octylacrylamide/methyl methacrylate/tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinylcaprolactam terpolymers and optionally derivatized cellulose ethers and silicones.

According to the invention, preference is given to the use of anionic polymers. These are used preferably in amounts of 0.1-5% by weight, preferably 0.1-3% by weight and in particular 0.1-2% by weight, based on the total composition. Polyacrylic acid homopolymers and copolymers are preferably suitable according to the invention. Particularly advantageous anionic polymers are those with the INCI name Carbomer, such as, for example, Carbopols of the types 980, 981, 1382, 2984, 5984 and also Rheocare C plus and Rheocare 400). Further advantageous anionic polymers are those with the INCI name Acrylates/C10-30 Alkyl Acrylate Crosspolymer (e.g. Pemulen TR, Pemulen TR 2, Carbopol 1328), Acrylates Copolymer (e.g. Rheocare TTA, TTN, TTN-2), Acrylamide/Sodium Acrylate Copolymer (e.g. Cosmedia ATC), Sodium Polyacrylate (e.g. Cosmedia ATH, SP), Polyacrylamide (e.g. Sepigel 305, 501).

Likewise suitable polymers are polysaccharides, in particular xanthan gum, guar guar, agar agar, alginates and tyloses and also, for example, Aerosil grades (hydrophilic silicas), carboxymethylcellulose and hydroxyethylcellulose and hydroxypropylcellulose, polyvinyl alcohol, polyvinylpyrrolidone and bentonites, such as, for example, Bentone® Gel VS-5PC (Rheox).

Likewise suitable are so-called quaternary polymers, e.g. with the INCI name Polyquaternium-37, which conform to the following general formula:

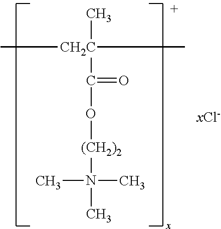

Alternatively, it is also possible to use other dialkylaminoalkyl (meth)acrylates and their ammonium salts or dialkylaminoalkyl(meth)acrylamides obtainable by alkylation or protonation, and also their ammonium salts obtainable by alkylation or protonation. Particular preference is given to polymers comprising MAPTAC, APTAC, MADAME, ADAME, DMAEMA and TMAEMAC. Moreover, it is also possible to use copolymers with anionic, further cationic or uncharged monomers in accordance with the invention, in particular those which, besides the specified alkylaminoalkyl (meth)acrylate or -(meth)acrylamide monomers, additionally comprise (meth)acrylic acid and/or 2-acrylamido-2-methylpropanesulfonic acid and/or acrylamide and/or vinylpyrrolidone and/or alkyl (meth)acrylates. By way of example, mention may be made of those polymers with the INCI name Polyquaternium-11, Polyquaternium-13, Polyquaternium-14, Polyquaternium-15, Polyquaternium-28, Polyquaternium-32, Polyquaternium-43, Polyquaternium-47.

Oil Bodies

In one embodiment of the invention, the preparations according to the invention comprise at least one oil body. Usually, the preparations according to the invention comprise n-octyl n-octanoate as oil body. In the embodiment specified here as preferred, the preparations thus comprise an oil body different from the n-octyl n-octanoate according to the invention, also referred to as “further oil body”. One subject matter of the invention relates to cosmetic and/or pharmaceutical preparations comprising 0.1 to 80% by weight of n-octyl n-octanoate and at least one (further) oil body. One preferred subject matter of the invention relates to cosmetic and/or pharmaceutical preparations comprising 0.1 to 80% by weight of n-octyl n-octanoate, and at least one (further) oil body.

The oil bodies (n-octyl n-octanoate according to the invention plus further oil bodies) are present usually in a total amount of 0.1-90, in particular 0.1-80, in particular 0.5 to 70, preferably 1 to 60, in particular 1 to 50% by weight, in particular 1 to 40% by weight, preferably 5-25% by weight and in particular 5-15% by weight. The further oil bodies are usually present in an amount of from 0.1 to 40% by weight—based on the total weight of the preparation.

Suitable further oil bodies are, for example, Guerbet alcohols based on fatty alcohols having 6 to 18, preferably 8 to 10, carbon atoms, and also further additional esters such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C₁₈-C₃₈-alkylhydroxycarboxylic acids with linear or branched C₆-C₂₂-fatty alcohols, in particular dioctyl malate, esters of linear and/or branched fatty acids with polyhydric alcohols (such as, for example, propylene glycol, dimerdiol or trimertriol), triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonate (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols and hydrocarbons or mixtures thereof. Also suitable as further oil bodies are hydrocarbons, such as, for example, n-undecane and/or n-tridecane, which are available under the trade name Cetiol®UT.

Suitable further oil bodies are, for example, silicone oils. They may be present as cyclic and/or linear silicone oils. Silicone oils are high molecular weight synthetic polymeric compounds in which silicon atoms are linked via oxygen atoms in a chain-like and/or grid-like manner and the remaining valences of silicon are saturated by hydrocarbon radicals (in most cases methyl, more rarely ethyl, propyl, phenyl groups etc.). Systematically, the silicone oils are referred to as polyorganosiloxanes. The methyl-substituted polyorganosiloxanes, which are the most important compounds of this group in terms of amount and are characterized by the following structural formula

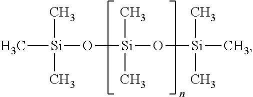

are also referred to as polydimethylsiloxane or dimethicone (INCI). Dimethicones come in various chain lengths and with various molecular weights.

Advantageous polyorganosiloxanes within the context of the present invention are, for example, dimethylpolysiloxane [poly(dimethylsiloxane)], which are available, for example, under the trade names Abil 10 to 10 000 from Evonik Goldschmidt. Also advantageous are phenylmethylpolysiloxane (INCI: Phenyl Dimethicone, Phenyl Trimethicone), cyclic silicones (octamethyl-cyclotetrasiloxane or decamethylcyclopentasiloxane), which are also referred to in accordance with INCI as cyclomethicone, amino-modified silicones (INCI: Amodimethicones) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Evonik Goldschmidt. However, other silicone oils can also be used advantageously within the context of the present invention, for example cetyldimethicone, hexamethylcyclotrisiloxane, polydimethylsiloxane, poly(methylphenylsiloxane). Silicones that are particularly preferred according to the invention are dimethicone and cyclomethicone.

The preparations according to the invention can also comprise biogenic active ingredients, insect repellents, tyrosinase inhibitors, preservatives, perfume oils, superfatting agents, stabilizers and/or hydrotropes.

One subject matter of the invention relates to cosmetic and/or pharmaceutical preparations comprising n-octyl n-octanoate and at least one biogenic active ingredient, insect repellent, tyrosinase inhibitor, preservative, perfume oil, stabilizer and/or hydrotrope.

A preferred subject matter of the invention relates to cosmetic and/or pharmaceutical preparations comprising 0.1 to 80% by weight of n-octyl n-octanoate and at least one biogenic active ingredient, insect repellent, tyrosinase inhibitor, preservative, perfume oil, stabilizer, fillers and/or hydrotrope.

Biogenic active ingredients are to be understood as meaning for example tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, such as e.g., aloe vera, prune extract, bambara nut extract and vitamin complexes. Suitable insect repellents are, for example, N,N-diethyl-m-toluamide, 1,2-pentanediol or ethyl 3-(N-n-butyl-N-acetylamino)propionate, which is sold under the name Insect Repellent® 3535 by Merck KGaA, and also butylacetylaminopropionates. Suitable tyrosine inhibitors, which prevent the formation of melanin and are used in depigmentation agents, are, for example, arbutin, ferulic acid, kojic acid, coumaric acid and ascorbic acid (vitamin C). Suitable preservatives are, for example, phenoxyethanol, formaldehyde solution, parabens, pentanediol or sorbic acid, and also the silver complexes known under the name Surfacine®. Furthermore, suitable preservatives are the 1,2-alkanediols having 5 to 8 carbon atoms described in WO07/048757. Suitable preservatives are in particular the substances approved according to Annex VI of the Commission Directive (in the version Commission Directive 2007/22/EC of 17 Apr. 2007 amending Council Directive 76/768/EEC, concerning cosmetic products, for the purposes of adapting Annexes IV and VI thereto to technical progress), to which reference is hereby explicitly made. Perfume oils which may be mentioned are mixtures of natural and synthetic fragrances. Natural fragrances are extracts from flowers, stems and leaves, fruits, fruit peels, roots, wood, herbs and grasses, needles and branches, resins and balsams. Also suitable are animal raw materials, such as, for example, civet and castoreum, and also synthetic fragrance compounds of the ester, ether, aldehyde, ketone, alcohol and hydrocarbon type. Stabilizers which can be used are metal salts of fatty acids, such as e.g. magnesium, aluminum and/or zinc stearate or ricinoleate. Suitable fillers are compounds which e.g. further improve the sensory and cosmetic properties of the preparations and, for example, bring about or enhance a velvety or silky skin feel (so-called skin sensory modifier). Within the context of the present invention, advantageous fillers are starch and starch derivatives (such as e.g. tapioca starch, distarch phosphate, aluminum or sodium starch octenylsuccinate and the like), pigments which have neither primarily a UV effect nor coloring effect (such as e.g. boron nitride etc.) and/or Aerosils® (CAS No. 7631-86-9) and/or talc, and also Polymethyl Methacrylate (e.g. Cosmedia® PMMA V8/V12), Silica (e.g. Cosmedia® SILC), Stearalkonium Hectorite (as contained e.g. in Cosmedia® Gel CC) and HDI/Trimethylol Hexyllactone Crosspolymer (as contained e.g. in Cosmedia® CUSHION). To improve the flow behavior, it is also possible to use hydrotropes, such as, for example, ethanol, isopropyl alcohol, or polyols. Polyols which are suitable here preferably have 2 to 15 carbon atoms and at least two hydroxyl groups. The polyols can also contain further functional groups, in particular amino groups, and/or be modified with nitrogen.

1. A cosmetic and/or pharmaceutical preparation comprising n-octyl n-octanoate.

2. The cosmetic and/or pharmaceutical preparation according to claim 1, comprising at least one antiperspirant/deodorant active ingredient.

3. The cosmetic and/or pharmaceutical preparation according to claim 1, comprising at least one UV photoprotective filter.

4. The cosmetic and/or pharmaceutical preparation according to claim 1, comprising at least one self-tanning agent.

5. The cosmetic and/or pharmaceutical preparation according to claim 1, comprising at least one pigment and/or dye.

6. The cosmetic and/or pharmaceutical preparation according to claim 1, comprising at least one emulsifier and/or a surfactant and/or a wax component and/or a polymer and/or a further oil body.

7. The cosmetic and/or pharmaceutical preparation according to claim 1, comprising 0.1 to 80% by weight of n-octyl n-octanoate.

8. The cosmetic and/or pharmaceutical preparation according to claim 2, comprising 0.1 to 80% by weight of n-octyl n-octanoate.

9. The cosmetic and/or pharmaceutical preparation according to claim 3 comprising 0.1 to 80% by weight of n-octyl n-octanoate.

10. The cosmetic and/or pharmaceutical preparation according to claim 4 comprising 0.1 to 80% by weight of n-octyl n-octanoate.

11. The cosmetic and/or pharmaceutical preparation according to claim 5 comprising 0.1 to 80% by weight of n-octyl n-octanoate.

12. The cosmetic and/or pharmaceutical preparation according to claim 6 comprising 0.1 to 80% by weight of n-octyl n-octanoate.

13. A method of using the cosmetic and/or pharmaceutical preparation according to claim 1 in cosmetic and/or pharmaceutical formulations.

14. The method of claim 13, wherein the cosmetic and/or pharmaceutical preparation comprises 0.1 to 80% by weight of n-octyl n-octanoate.
